(19) United States
(12) Patent Application Publication
(10) Pub. No.: US 2025/0312426 A1
(43) Pub. Date: Oct. 9, 2025

(54) NOVEL COMPOSITIONS AND USES THEREOF FOR PRODUCTION OF ALLERGEN TOLERANCE

(71) Applicant: Aadi Gannavaram, Potomac, MD (US)

(72) Inventor: Aadi Gannavaram, Potomac, MD (US)

(21) Appl. No.: 18/626,462

(22) Filed: Apr. 4, 2024

(51) Int. Cl. A61K 39/00; A61P 37/08

(52) U.S. Cl. CPC A61K 39/0008; A61P 37/08; A61K 2039/54; A61K 2039/55555

(57) ABSTRACT

The present disclosure provides compositions for inducing immune tolerance to one or more allergens in a subject. The compositions typically comprise an implantable device, such as an intradermal implantable device, designed to deliver one of more specific antigens (allergens) and aryl hydrocarbon receptor (AhR) agonists. The invention also provides methods for inducing tolerance to a protein or polypeptide antigen (e.g., an allergen) in a subject. The methods involve administering to the subject a pharmaceutical composition that co-presents both the antigen and aryl hydrocarbon receptor (AhR) agonists.

### FIELD OF THE INVENTION

The present disclosure provides pharmaceutical compositions for inducing immune tolerance to one or more allergens in a subject. The pharmaceutical compositions typically comprise one of more specific antigens (allergens) and an aryl hydrocarbon receptor (AhR) agonists for delivery via, for example, an implantable device such as an intradermal implantable device. The invention also provides methods for inducing tolerance to an antigen (e.g., an allergen) in a subject. The methods involve administering to the subject a pharmaceutical composition that co-presents both the antigen and an AhR agonists.

### BACKGROUND OF THE INVENTION

Food allergies affect 32 million Americans and result in adverse quality of life and psychological burden. The only FDA approved peanut oral immunotherapy showed the potential of immunotherapies, however novel strategies with better therapeutic outcomes towards inducing tolerance are widely sought. Immunoglobulin E mediated allergic reaction occur when basophils, eosinophils release pro-inflammatory mediators such as histamine. Tolerance to allergens is developed when regulatory T cells that produce TGF-β and interleukin-10 counteract allergic inflammation. Aryl hydrocarbon receptor (AhR) is a transcription factor that when activated produces TGF-β and IL-10.

Food allergic reactions result from the immune system producing compounds that trigger excessive physiological responses. One of the most common types of allergy is mediated by immunoglobulin E (IgE), an antibody that is overproduced in response to an allergen (Akdis et al., 2023 Sci Transl Med. 18:679). IgE binds to mast cells, found in the connective tissue around the body, which then produce histamines in a process called degranulation, the compounds responsible for the physiological allergic reaction. The most commonly used allergy suppression methods today rely on the direct use of biologicals, in this case, compounds to stop anaphylaxis or antihistamines. Anaphylaxis is a potentially lethal allergic reaction in which the overproduction of histamines as an immune response to the allergen leads to the patient going into shock, losing blood pressure, and struggling to breathe. This reaction is handled using biologicals such as epinephrine, also known as adrenaline. It is delivered through auto-injection devices, including products such as EpiPen. Epinephrine is able to alter ion channel modulation in myocardial pacemaker cells (Ju and Allen, 1999 J Physiol 516:793-804), thereby increasing the heart rate and causing smooth muscles in the vascular system to contract, increasing blood pressure. Epinephrine also relaxes smooth bronchial muscle cells (Baldwin et al., 1994, Thorx 49:1103-8) preventing suffocation. While this is an immediate response, it is only preferable when the patient is in a life-threatening state. It is not a prophylactic solution and must be taken after the reaction has already occurred and does not directly counteract the histamines.

Currently, antihistamine drugs are used to directly mitigate the production of histamines from mast cells. Histamines cause allergic reactions by binding to H1 receptors found on cells in cardiovascular and endothelial cells, which causes inflammation. Antihistamines, such as Diphenhydramine (found in Benadryl) bind to the H1 receptors and physically block access from the histamines. This removes symptoms by mitigating the direct effects of H1 receptor binding but is still not prophylactic and is used after the full symptoms have already appeared. Other common treatments for allergic diseases include antihistamines, corticosteroids, leukotriene receptor antagonists, and Janus Kinase inhibitors (Lee et al., 2023).

A more novel approach is through allergen-specific immunotherapy, which is treatment via amplification of an immune response. Allergen immunotherapy aims to repeatedly expose the patient to the allergen, which allows the creation of regulatory T cells (TReg cells) that produce cytokines such as interleukin-10 (IL-10) and transformation growth factor-β (TGF-β) (Akdis et al., 2023 Sci Transl Med. 18:679). These cytokines suppress production of inflammatory cytokines in macrophages and dendritic cells (Iyer and Cheng 2012 Crit Rev Immunol 32:23-63), and mast cell degranulation (Polukort et al., 2016 J Immunol 196:4865-76), thereby halting the symptoms and any continuation of the allergic reaction. Different ways are being developed to implement this clinically. Subcutaneous immunotherapy is a common form of this type of treatment, in which the patient receives under-skin injections of small doses of the allergen at regular intervals. This is similar to oral immunotherapy, in which the patient ingests small solid amounts of the allergen at regular intervals with the hope of slowly building immunity. Sublingual Immunotherapy Tablets (SLIT tablets) have emerged as a safer alternative in recent years, where small, powdered doses of the allergen are contained in pills that dissolve under the patient's tongue. However, as all the aforementioned methods do not directly amplify the immune response to the allergen, the doses must be small enough to avoid threat to the patient. This renders the prophylatic development processes very slow, taking three to five years minimum for all the methods. Accordingly, there is currently a need for novel methods for inducing tolerance to allergens in a subject.

### SUMMARY OF THE INVENTION

It has been discovered that tolerance to allergens is developed when regulatory T cells that produce TGF-β and interleukin-10 counteract allergic inflammation. Aryl hydrocarbon receptor (AhR) is a transcription factor that, when activated, stimulates production of TGF-β and IL-10. Accordingly, the present disclosure provides pharmaceutical compositions and methods towards inducing tolerance efficiently in a subject based on, for example, implantable multiplex devices that release one or more allergens in combination with AhR agonists in a controlled manner to elicit a robust T reg cell response resulting in development of tolerance to said allergens in a subject.

In an embodiment, the present disclosure provides pharmaceutical compositions for inducing immune tolerance to specific antigens. The pharmaceutical compositions will typically contain one or more specific antigens and also at least one AhR agonist. The specific antigen against which immune tolerance is to be induced can be a protein. The antigen can also be a hapten, a carbohydrate, or a nucleic acid. In some embodiments the pharmaceutical compositions are directed to use in inducing immune tolerance to an autoantigen, an allergen, or an alloantigen. In a non-limiting embodiment, an implantable device is provided, comprising the provided pharmaceutical compositions, wherein said device permits the well-controlled release of antigen and AhR agonists into the subject.

In a related aspect, the present disclosure provides methods for inducing tolerance to an antigen in a subject (e.g. a specific allergen). These methods entail administering to the subject a pharmaceutical composition that contains an effective amount of the antigen and an AhR agonists in a controlled manner through the use of, for example, an intradermal implantable device. In related aspects, kits comprising the disclosed pharmaceutical compositions are also provided for induction of allergen tolerance. Such kits contain materials useful for the methods of inducing tolerance to an allergen as described herein.

### BRIEF DESCRIPTION OF THE DRAWINGS

FIG. 1. The production of a tolerogenic condition by efficiently inducing T regulatory cells.

FIG. 2A-F. Expression of regulatory cytokines in co-cultures of murine dendritic cells and T lymphocytes. The dendritic cell and T lymphocyte coculture system is an in vitro model to test the effect of various metabolites in conjunction with food allergens. Bone marrow derived dendritic cells were cocultured with T lymphocytes in presence or absence of AhR ligands 6-Formylindolo[3,2-b]carbazole (FICZ) or Kynurenine (Kyn) or Kynurenic acid (KynA) for 48 hours. Total RNA was isolated from the non-adherent T lymphocytes and RT-PCR was performed from the cDNA. Expression of regulatory cytokines was compared to the housekeeping gene β-actin. Expression of IL-10 (FIG. 2A) and the transcription factor AhR (FIG. 2B) in presence or absence of exogenously added FICZ are shown. Expression of TGF-β (FIG. 2C) and the rate-limiting enzyme for tryptophan metabolism IDO1 (FIG. 2D) in presence or absence of exogenously added Kynurenine are shown. Expression of TGF-β (FIG. 2E) and the rate-limiting enzyme for tryptophan metabolism IDO1 (FIG. 2F) in presence or absence of exogenously added Kynurenic acid are shown. Supplementation of tryptophan metabolites in presence of food allergens will similarly induce the production of regulatory cytokines.

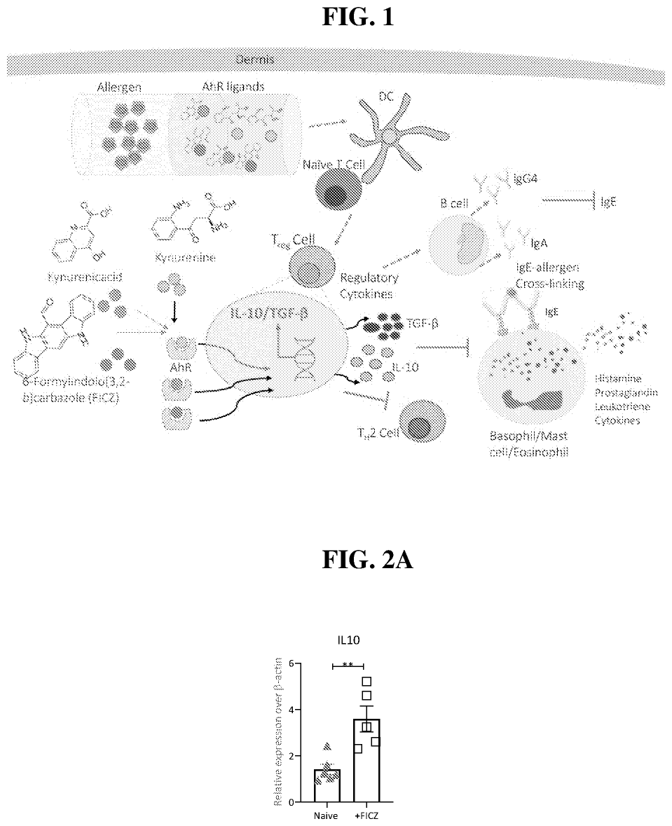

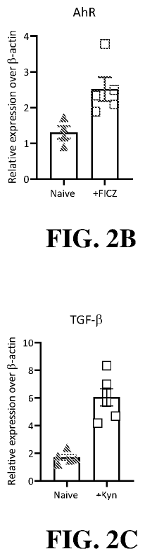

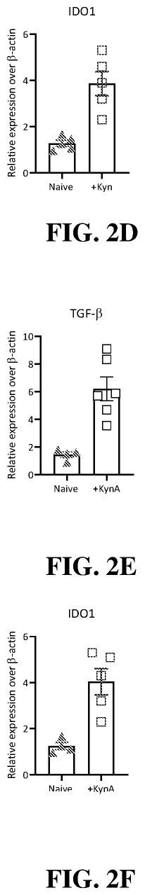

### DETAILED DESCRIPTION OF THE INVENTION

The following is a detailed description of the novel pharmaceutical compositions and methods for induction of tolerance to an antigen (allergen) in a subject. Unless otherwise defined, all technical and scientific terms used herein have the same meaning as commonly understood by one of ordinary skill in the art to which this invention belongs. The terminology used in the present disclosure is for describing particular embodiments only and is not intended to be limiting of the disclosure. All publications mentioned herein are incorporated herein by reference to disclose and described the methods and/or materials in connection with which the publications are cited.

As used herein, immune tolerance (or simply “tolerance”) is the process by which the immune system does not attack an antigen.

As used herein, “antigen” and “allergen” are used interchangeably herein to refer to a molecule capable of eliciting an allergic reaction or rejection of tissue.

The term “subject” as used herein refers to an animal. Typically, the animal is a mammal. A subject also refers to for example, primates (e.g., humans), cows, sheep, goats, horses, dogs, cats, rabbits, rats, mice, fish, birds and the like. In certain embodiments, the subject is a primate. In yet other embodiments, the subject is a human.

The terms “effective amount” or “therapeutically effective amount” as used herein have the standard meanings known in the art and are used interchangeably herein to mean an amount sufficient to treat a subject afflicted with a condition or disease (e.g., an allergic reaction to contact with an allergen, autoimmune diseases) or to halt the progression of the condition or disease, or alleviate a symptom or a complication associated with the condition or disease. The exact dose will depend on the purpose of the treatment, and will be ascertainable by one skilled in the art using known techniques (e.g., Ansel et al., Pharmaceutical Dosage Forms and Drug Delivery; Lieberman, Pharmaceutical Dosage Forms (vols. 1-3, 1992), Dekker, ISBN 0824770846, 082476918X, 0824712692, 0824716981; Lloyd, The Art, Science and Technology of Pharmaceutical Compounding (1999); and Pickar, Dosage Calculations (1999)). For example, in the case of an agent to treat allergies or autoimmune disease, an effective amount may be an amount sufficient to result in clinical improvement of the patient.

The terms “protein” and “polypeptide” as used herein are used interchangeably, unless specified to the contrary, and according to conventional meaning, mean a sequence of amino acids. Peptides are not limited to a specific length, e.g., they may comprise a full-length protein sequence or a fragment of a full-length protein, and may include post-translational modifications of the polypeptide, for example, glycosylations, acetylations, phosphorylations and the like, as well as other modifications known in the art, both naturally occurring and non-naturally occurring, e.g., variants. In an embodiment, a polyeptide may comprise an epitope that is responsible for eliciting an allergic reaction.

The term “therapeutic agent” as used herein is a compound capable of producing a desired and beneficial effect, e.g., stimulation of tolerance to a select allergen.

The terms “treat,” “treating” or “treatment” of any disease or disorder as used herein refer in one embodiment, to halting the progression of the condition or disease, or to ameliorating the disease or disorder (i.e., slowing or arresting or reducing the development of the disease or at least one of the clinical symptoms thereof). In another embodiment “treat,” “treating” or “treatment” refers to alleviating or ameliorating at least one physical parameter including those which may not be discernible by the patient. In yet another embodiment, “treat,” “treating” or “treatment” refers to modulating the disease or disorder, either physically, (e.g., stabilization of a discernible symptom), physiologically, (e.g., stabilization of a physical parameter), or both. In yet another embodiment, “treat,” “treating” or “treatment” refers to preventing or delaying the onset or development or progression of the disease or disorder. As used herein, a subject is “in need of” a treatment if such subject would benefit biologically, medically or in quality of life from such treatment.

It has been discovered that tolerance to allergens is developed when regulatory T cells that produce TGF-β and interleukin-10 counteract allergic inflammation. Aryl hydrocarbon receptor (AhR) is a transcription factor that, when activated, produces TGF-β and IL-10. Accordingly, the present disclosure provides pharmaceutical compositions, devices and methods for inducing tolerance efficiently in a subject based on the release of one or more allergens, in combination with at least one AhR agonists, in a controlled manner to elicit a robust T reg cell response resulting in development of tolerance to said allergens in the subject.

The pharmaceutical compositions typically contain one of more specific antigens and also at least one AhR agonists. Various antigens can be used in preparing the pharmaceutical compositions for inducing immune tolerance as disclosed herein. In some embodiments, the antigen is one that is involved in an undesired immune reaction or response in a subject exposed to said antigen. In some embodiments, the employed antigen is any antigen to which a subject may be at risk of developing an undesired immune reaction or response.

Antigens of different chemical nature are suitable for inducing immune tolerance in a subject. They include polypeptides or proteins, haptens, carbohydrates, nucleic acids, peptides, polyethylene glycol, lipids, polysaccharides, and gangliosides. In additional embodiments, the provided pharmaceutical compositions and methods are directed to inducing immune tolerance to allergens. For example, various allergens from food, pollen, mold, pet dander, and dust mites are suitable for practice of the invention. This includes, for example, nut allergens; milk allergens; wheat gluten allergens, fruit allergens and fish/shellfish allergens. In some embodiments, the employed allergen is a latex allergen. In addition, allergens may include, but not limited to, grass allergens, major dust mite allergens, domestic cat allergens, and tree, grass and ragweed pollen allergens.

In some embodiments the provided methods are intended for inducing immune tolerance to various autoantigens. Autoantigens are known for a number of autoimmune diseases. In some embodiments, the provided methods are directed to inducing tolerance against protein antigens that are normally self-antigens, but which certain individuals lack resulting from a genetic deficiency and to which an unwanted immune reaction occurs upon replacement therapy.

In some embodiments, the employed antigen for inducing immune tolerance is an alloantigen. Alloantigens are generally cellular antigens that vary in structure among individual members of a single species. Alloantigens from one individual can be recognized as foreign antigens by other members of the same species and are often the basis for graft rejection reactions. Examples of alloantigens include, but are not limited to, major histocompatability complex (MHC) class I and class II antigens, minor histocompatability antigens, certain tissue-specific antigens, endothelial glycoproteins such as blood group antigens, and carbohydrate determinants.

In an embodiment, the allergen to be co-administered with the AhR agonist is pre-selected based on results obtained using an allergy test. Such allergy tests include, for example, allergy skin tests, where skin is exposed to suspected allergy-causing substances (allergens) and is then observed for signs of an allergic reaction. Skin testing is usually done at a doctor's office. A nurse generally administers the test, and a doctor interprets the results. Typically, this test takes about 20 to 40 minutes. Some tests detect immediate allergic reactions, which develop within minutes of exposure to an allergen. Other tests detect delayed allergic reactions, which develop over a period of several days. A skin prick test, also called a puncture or scratch test, checks for immediate allergic reactions to as many as 50 different substances at once. This test is usually done to identify allergies to pollen, mold, pet dander, dust mites and foods. In adults, the test is usually done on the forearm. Children may be tested on the upper back. Patch testing is generally done to see whether a particular substance is causing allergic skin inflammation (contact dermatitis). Patch tests can detect delayed allergic reactions, which can take several days to develop. Blood tests (in vitro immunoglobulin E antibody tests) can also be useful for those who shouldn't or can't undergo skin tests.

The various antigens suitable for practicing the provided methods may be isolated from their source using purification techniques known in the art or, more conveniently, may be produced using recombinant methods. For example, the antigens can be obtained through a number of methods known in the art, including isolation and synthesis using chemical and enzymatic methods. In certain cases, the antigenic portions of the molecules are commercially available. Antigens derived from infectious agents may be obtained using methods known in the art, for example, from viral or bacterial extracts, from cells infected with the infectious agent, from purified polypeptides, from recombinantly produced polypeptides and/or as synthetic peptides.

Methods of producing antigenic proteins, polypeptide fragments or variants thereof, for use in the methods disclosed herein may be made in a variety of ways. For example, solid phase synthesis techniques may be used. Suitable techniques are well known in the art, and include those described in Merrifield, in Chem. Polypeptides, pp. 335-61 (Katsoyannis and Panayotis eds. 1973); Merrifield, J. Am. Chem. Soc. 85:2149 (1963); Davis et al., Biochem. Intl. 10:394-414 (1985); Stewart and Young, Solid Phase Peptide Synthesis (1969); U.S. Pat. No. 3,941,763; Finn et al., The Proteins, 3rd ed., vol. 2, pp. 105-253 (1976); and Erickson et al., The Proteins, 3rd ed., vol. 2, pp. 257-527 (1976). Solid phase synthesis is the preferred technique of making individual peptides since it is the most cost-effective method of making small peptides that may serve as antigens. The antigenic proteins, polypeptide fragments or variants thereof, for use in the methods disclosed herein may also be made using recombinant DNA techniques which are well known in the art. See, for example, the techniques described in Maniatis et al., MOLECULAR CLONING: A LABORATORY MANUAL, Cold Spring Harbor Laboratory, N.Y. (1989); and Ausubel et al., CURRENT PROTOCOLS IN MOLECULAR BIOLOGY, Greene Publishing Associates and Wiley Interscience, N.Y. (1989).

Aryl hydrocarbon receptors (AhR) are transcription factors found in immune cells such as myeloid cells, innate lymphoid cells, B lymphocytes and certain subtypes of T cells. Aryl hydrocarbon receptors, when activated, inhibit the release of pro-inflammatory proteins (cytokines) by immune cells and downregulate inflammation. AhR agonists work by binding to and activating aryl hydrocarbon receptors on the skin cells and immune cells that cause inflammation.

AhR agonists that may be used in the provided methods for induction of tolerance to an antigen include, but are not limited to, Tapinarof, FICZ, 6-formylindolo (3,2-b) carbazole; ICZ, indolo (3,2-b) carbazole; ITE, 2-(1′H-indole-3′-carbonyl)-thiazole-4-carboxylic acid methyl ester; PAH, polycyclic aromatic hydrocarbon; PCB, polychlorinated biphenyl; TCDD, 2,3,7,8 tetrachlorodibenzo-p-dioxin; 3,9-dibromotryptanthrin; 5,5′-dichloroindirubin; 6,6′-dibromoindirubin; 3-bromotryptanthrin; (1H-indol-3-yl)(6-(trifluoromethyl)pyridin-2-yl)methanone and 6-(1H-indole-3-carbonyl)picolinonitrile. Prototypical AHR agonists include halogenated dibenzodioxins such as 2,3,7,8-tetrachlorodibenzodioxin (TCDD), tryptophan metabolites such as L-kynurenine, bilirubin, carbidopa, microbial-derived 1,4-dihydroxy-2-napthoic acid, plant-derived 3,3-diindolylmethane, benzo-a-pyrene (BaP), indirubin, pentaCB (PCB), lipoxin A₄ and PGE2.

Agonists of AhR include the halogenated aromatic hydrocarbons (polychlorinated dibenzodioxins, dibenzofurans and biphenyls) and polycyclic aromatic hydrocarbons (3-methylcholanthrene, benzo[a]pyrene, benzanthracenes and benzoflavones). Also included are derivatives of tryptophan such as indigo dye and indirubin, tetrapyrroles such as bilirubin, the arachidonic acid metabolites lipoxin A4 and prostaglandin G, modified low-density lipoprotein and several dietary carotenoids.

Useful pharmaceutical compositions for delivery of the one or more antigens and at least one AhR agonists include biodegradable microcapsules or liposomes. Such liposomes may be unilamellar or multilamellar vesicles, having a membrane portion formed of lipophilic material and an interior aqueous portion. Pharmaceutical compositions provided herein also include those comprising synthetic nanocarriers that release a particular amount of the one or more antigens and at least one AhR agonists within a particular time period. In one aspect, the biodegradable microcapsule, liposomes or nanocarriers may be designed for controlled sloe release of the antigens and AhR agonists into the subject. In embodiments, the pharmaceutical compositions described herein are pharmaceutical compositions comprising synthetic nanocarriers to which the one or more antigens and at least one AhR agonists are bound.

In one embodiment, the synthetic nanocarrier comprises lipid nanoparticles, polymer nanoparticles, metal nanoparticles, surfactant-based emulsions, dendrimers, buckyballs, nanowires, virus-like particles or peptide or protein particles. In one embodiment, the synthetic nanocarrier comprises lipid nanoparticles. In another embodiment, the synthetic nanocarrier comprises a liposome.

In one aspect, a nanoparticle comprising the antigen and AhR agonist as disclosed herein is provided. Such nanoparticles can be natural or synthetic. They can be created from biological molecules or from non-biological molecules. In some cases, the antigen and AhR agonist is crosslinked to a polymer or lipid on the nanoparticle surface. In embodiments, the antigen and AhR agonist is adsorbed onto the nanoparticle surface. In some embodiments, the antigen and AhR agonist is adsorbed onto the nanoparticle surface and then crosslinked to the nanoparticle surface. In some embodiments, the antigen and AhR agonist is encapsulated into the nanoparticle.

In particular embodiments, the nanoparticle is formed from a biocompatible polymer. Examples of biocompatible polymers include polyethylenes, polycarbonates, polyanhydrides, polyhydroxyacids, polypropylfumerates, polycaprolactones, polyamides, polyacetals, polyethers, polyesters, poly (orthoesters), polycyanoacrylates, polyvinyl alcohols, polyurethanes, polyphosphazenes, polyacrylates, polymethacrylates, polycyanoacrylates, polyureas, polystyrenes, or polyamines, or combinations thereof. In some cases, the nanoparticle is formed from a polyethylene glycol (PEG), poly(lactide-co-glycolide) (PLGA), polyglycolic acid, poly-beta-hydroxybutyrate, polyacrylic acid ester, or a combination thereof.

In a specific embodiment the nanoparticle is a nanoliposome. Such nanoliposomes may be composed of phospholipids such as 1,2-distearoyl-sn-glycero-3-phosphocholine (DSPC), 1,2-dipalmitoyl-sn-glycero-3-phosphocholine (DPPC), 1,2-dimyristoyl-sn-glycero-3-phosphocholine (DMPC), 1,2-dioleoyl-sn-glycero-3-phosphocholine (DOPC), 1,2-distearoyl-sn-glycero-3-phospho-(1′-rac-glycerol) (DSPG), 1,2-dipalmitoyl-sn-glycero-3-phospho-(1′-rac-glycerol) (DPPG), 1,2-dimyristoyl-sn-glycero-3-phospho-(1′-rac-glycerol) (DMPG), 1,2-dioleoyl-sn-glycero-3-phospho-(1′-rac-glycerol) (DOPG), dipalmitoyl phosphatidylserine (DPPS), distearoyl phosphatidylserine (DSPS), dipalmitoyl phosphatidylinositol (DPPI), distearoyl phos phatidylinositol (DSPI), dipalmitoyl phosphatidic acid (DPPA), distearoyl phosphatidic acid (OSPA), 1,2-diacyl-3-trimethylammonium-propanes, (including but not limited to, dioleoyl (DOTAP), 1,2-dipalmitoyl-sn-glycero-3-phosphoethanolamine-N [methoxy(polyethylene glycol)-2000] (DPPE-PEG2000), 1,2-distearoyl-sn-glycero-3-phosphoethanolamine-N-[methoxy(polyethylene glycol)-1000] (DSPE-PEG2000), and cholesterol.

The pharmaceutical composition formulations may be designed to be short-acting, fast-releasing, long-acting, or sustained-releasing as described herein. In one aspect, the formulations are designed for controlled sustained-releasing.

Pharmaceutical compositions of embodiments comprise a therapeutically effective amount of one or more antigens and an AhR agonist dissolved or dispersed in a pharmaceutically acceptable carrier. The preparation of a pharmaceutical composition that contains at one or more antigens and an AhR agonist and optionally an additional active ingredient will be known to those of skill in the art in light of the present disclosure, as exemplified by Remington's Pharmaceutical Sciences, 18th Ed. Mack Printing Company, 1990. For human administration, it will be understood that preparations should meet sterility, pyrogenicity, general safety and purity standards as required by FDA Office of Biological Standards or corresponding authorities in other countries. Preferred pharmaceutical compositions are lyophilized formulations or aqueous solutions.

As used herein, “pharmaceutically acceptable carrier” includes any and all solvents, buffers, dispersion media, coatings, surfactants, antioxidants, preservatives (e.g. antibacterial agents, antifungal agents), isotonic agents, absorption delaying agents, salts, preservatives, antioxidants, proteins, drugs, drug stabilizers, polymers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference). Except insofar as any conventional carrier is incompatible with the active ingredient, its use in the pharmaceutical compositions is contemplated.

The pharmaceutical composition may comprise different types of carriers depending on whether it is to be administered in solid, liquid or aerosol form, and whether it needs to be sterile for such routes of administration as injection. Antigens and AhR agonists of certain embodiments (and any additional therapeutic agent) can be administered by any method or any combination of methods as would be known to one of ordinary skill in the art (see, for example, Remington's Pharmaceutical Sciences, 18th Ed. Mack Printing Company, 1990, incorporated herein by reference). Parenteral administration, in particular intravenous injection, may be used for administering protein or polypeptide molecules such as the one or more antigens and AhR agonists of certain embodiments. Aqueous injection suspensions may contain compounds which increase the viscosity of the suspension, such as sodium carboxymethyl cellulose, sorbitol, dextran, or the like. Optionally, the suspension may also contain suitable stabilizers or agents which increase the solubility of the compounds to allow for the preparation of highly concentrated solutions. Additionally, suspensions of the active compounds may be prepared as appropriate oily injection suspensions. Suitable lipophilic solvents or vehicles include fatty oils such as sesame oil, or synthetic fatty acid esters, such as ethyl cleats or triglycerides, or liposomes.

Parenteral pharmaceutical compositions include those designed for administration by injection, e.g. subcutaneous, intradermal, intra-lesional, intravenous, intra-arterial, intramuscular, intrathecal or intraperitoneal injection. For injection, the one or more antigens and AhR agonists may be formulated in aqueous solutions, preferably in physiologically compatible buffers such as Hanks' solution, Ringer's solution, or physiological saline buffer. The solution may contain formulatory agents such as suspending, stabilizing and/or dispersing agents. Alternatively, the one or more antigens and AhR agonists may be in powder form for reconstitution with a suitable vehicle, e.g., sterile pyrogen-free water, before use. Sterile injectable solutions are prepared by incorporating the one or more antigens and AhR agonists in the required amount in the appropriate solvent with various other ingredients enumerated below, as required. Sterility may be readily accomplished, e.g., by filtration through sterile filtration membranes. Generally, dispersions are prepared by incorporating the various sterilized active ingredients into a sterile vehicle which contains the basic dispersion medium and/or the other ingredients. In the case of sterile powders for the preparation of sterile injectable solutions, suspensions or emulsion, the preferred methods of preparation are vacuum-drying or freeze-drying techniques which yield a powder of the active ingredient plus any additional desired ingredient from a previously sterile-filtered liquid medium thereof. The liquid medium should be suitably buffered if necessary and the liquid diluent first rendered isotonic prior to injection with sufficient saline or glucose. The pharmaceutical composition must be stable under the conditions of manufacture and storage, and preserved against the contaminating action of microorganisms, such as bacteria and fungi.

Pharmaceutical compositions comprising one or more antigens and AhR agonists may be manufactured by means of conventional mixing, dissolving, emulsifying, encapsulating, entrapping or lyophilizing processes. Pharmaceutical compositions may be formulated in conventional manner using one or more physiologically acceptable carriers, diluents, excipients or auxiliaries which facilitate processing of the proteins into preparations that can be used pharmaceutically. Proper formulation is dependent upon the route of administration chosen.

The pharmaceutical preparation of certain embodiments is a liquid pharmaceutical composition, e.g. an aqueous solution. For injection purposes, the use of pure water as solvent is preferred. Other solvents which are suitable and conventional for pharmaceutical preparations can, however, also be employed. In a preferred embodiment, the pharmaceutical compositions are isotonic solutions. Further, there is no need for reconstitution at any stage of the preparation of the liquid solution formulation of these embodiments. The solution is a ready-to-use formulation.

In a preferred aspect, controlled sustained-release preparations may be prepared. Suitable examples of sustained-release preparations include semipermeable matrices of solid hydrophobic polymers containing the antigen and AhR agonist, which matrices are in the form of shaped articles, e.g., films, or microcapsule. Examples of sustained-release matrices include polyesters, hydrogels (for example, poly (2-hydroxyethyl-methacrylate), or poly (vinyl alcohol)), polylactides (U.S. Pat. No. 3,773,919), copolymers of L-glutamic acid and y ethyl-L-glutamate, non-degradable ethylene-vinyl acetate, degradable lactic acid-glycolic acid copolymers (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods. When encapsulated polypeptides, e.g., antigens, remain in the body for a long time, they may denature or aggregate as a result of exposure to moisture at 37° C., resulting in a loss of biological activity and possible changes in immunogenicity. Rational strategies can be devised for stabilization depending on the mechanism involved. For example, if the aggregation mechanism is discovered to be intermolecular S—S bond formation through thio-disulfide interchange, stabilization may be achieved by modifying sulfhydryl residues, lyophilizing from acidic solutions, controlling moisture content, using appropriate additives, and developing specific polymer matrix pharmaceutical compositions.

In one aspect, a transdermal patch which is a medicated adhesive patch that is placed on the skin to deliver a specific dose of medication through the skin and into the bloodstream may be used to deliver the one or more antigens and the at least one AhR agonist. An advantage of a transdermal drug delivery route over other types of medication delivery (such as oral, topical, intravenous, or intramuscular) is that the patch provides a controlled release of the medication into the patient, usually through either a porous membrane covering a reservoir of medication or through body heat melting thin layers of medication embedded in the adhesive. The main disadvantage to transdermal delivery systems results from the fact that the skin is a very effective barrier and as a result, only medications whose molecules are small enough to penetrate the skin can be delivered by this method. In order to overcome restriction from the skin, microneedle transdermal patches (MNPs) have been developed which consist of an array of microneedles, which allows a more versatile range of compounds or molecules to be passed through the skin. Also, with advances in this technology, many such patches have been made multifunctional by incorporating smart components including electrothermal actuators, miniaturized physiological sensors for feedback therapy, self-powered (transdermal) systems deriving energy from human motion and wirelessly-controlled communication modules for tunable on-demand drug delivery.

In addition to patches, devices which are surgically placed inside body tissue may be used to deliver the one or more antigens and the at least one AhR agonist to the body of the subject, thereby providing for an extended delivery duration. Such devices include, for example, micropumps, and hydrogel/nanofibrous scaffolds. Injectable hydrogels, in situ gels, micropumps, shape-memory materials, are some of the delivery devices that have the potential to be implanted using minimally invasive procedures.

Accordingly, the present disclosure provides methods for inducing tolerance to an allergen in a subject comprising administering to the subject, an effective amount of an antigen and an AhR agonist in a pharmaceutically acceptable form. The present disclosure provides methods and pharmaceutical composition for treatment of, for example, allergies and autoimmune diseases by administering an antigen and an AhR agonist to a subject in need. Such auto-immune diseases include, but are not limited to ankylosing spondylitis, lupus, rheumatoid arthritis, juvenile arthritis, scleroderma dermatomyositis, behcet's disease, reactive arthritis, mixed connective tissue disease, raynaud's phenomenon, giant cell arteritis/temporal arteritis, polymyalgia rheumatica, polyarteritis nodosa, polymyositis, takayasu arteritis, granulomatosis with polyangiitis, and vasculitis, alopecia areata, antiphospholipid antibody syndrome, autoimmune hepatitis, type 1 diabetes, celiac disease, Chron's disease, Graves' disease, Guillain-Barre syndrome, Hashimoto's disease, idiopathic thrombocytopeniaurpura, inflammatory bowel disease, multiple sclerosis, myasthenia gravis, primary biliary cirrhosis, psoriasis, Sjogren's syndrome, vitiligo, bullous pemphigoid, pemphigus foliaceus, pemphigus vulgaris and epidermolysis bullosa acquisita.

Any of the antigens and AhR agonists provided herein may be used in therapeutic methods described herein. For use in the therapeutic methods described herein, one or more antigens and AhR agonists of certain embodiments would be formulated, dosed, and administered in a fashion consistent with good medical practice. The dosage form or pharmaceutical composition may be administered by intravenous, transmucosal, intraperitoneal, oral, subcutaneous, transpulmonary, intranasal, intradermal, or intramuscular administration. In a preferred aspect, the composition is administered intradermally employing a device for controlled sustained release of the antigen and AhR agonists.

Factors for consideration in this context include the allergy being treated, the particular subject being treated, the clinical condition of the subject, the cause of the allergy or condition, the site of delivery of the agent, the method of administration, the scheduling of administration, and other factors known to medical practitioners or those of skill in the art.

For the treatment of allergies or auto-immune disorders, the appropriate dosage of the antigen and AhR agonist will depend on the type of disease to be treated, the route of administration, the body weight of the patient, the severity and course of the disease, and whether the antigen and AhR agonist is co-administered for preventive or therapeutic purposes, previous or concurrent therapeutic interventions, the patient's clinical history and response to the administration of the antigen and AhR agonist, and the discretion of the attending physician. The practitioner responsible for administration will, in any event, determine the concentration of active ingredient(s) in a pharmaceutical composition and appropriate dose(s) for the individual subject. Various dosing schedules including but not limited to single or multiple administrations over various time-points, bolus administration, and pulse infusion are contemplated herein.

The antigen and AhR agonist are suitably administered to the patient at one time or over a series of treatments subcutaneously, intradermally, intravenously or intramuscularly. For repeated administrations over several days or longer, depending on the condition, the treatment would generally be sustained until a desired suppression of allergy or auto-immune disease symptoms occurs. Such doses may be administered intermittently, e.g. every week or every three weeks. An initial higher loading dose, followed by one or more lower doses may be administered. However, other dosage regimens may be useful. The progress of this therapy is easily monitored by conventional techniques and assays. The administration of the antigen and AhR agonist of certain embodiments will generally be used in an amount effective to achieve the intended purpose. The determination of a therapeutically effective amount is well within the capabilities of those skilled in the art, especially in light of the detailed disclosure provided herein.

For systemic administration, a therapeutically effective dose can be estimated initially from in vitro assays, such as cell culture assays. A dose can then be formulated in animal models to achieve a circulating concentration range that includes the IC₅₀ as determined in cell culture. Such information can be used to more accurately determine useful doses in humans. Initial dosages can also be estimated from in vivo data, e.g., animal models, using techniques that are well known in the art. One having ordinary skill in the art could readily optimize administration to humans based on animal data.

The antigen and AhR agonist containing pharmaceutical compositions may be administered by a continuous infusion to maintain therapeutic circulating levels of antigen and AhR agonist. Those of ordinary skill in the art will readily optimize effective dosages and administration regimens as determined by good medical practice and the clinical condition of the individual patient.

The attending physician for patients treated with pharmaceutical compositions disclosed herein would know how and when to terminate, interrupt, or adjust administration due to toxicity, organ dysfunction, and the like. Conversely, the attending physician would also know to adjust treatment to higher levels if the clinical response were not adequate (precluding toxicity). The magnitude of an administered dose in the management of the disorder of interest will vary with the severity of the condition to be treated, with the route of administration, and the like. The severity of the condition may, for example, be evaluated, in part, by standard prognostic evaluation methods. Further, the dose and perhaps dose frequency will also vary according to the age, body weight, and response of the individual patient.

In another aspect of the embodiment, an article of manufacture (e.g., a kit) containing materials useful for the treatment of allergies and autoimmune disorders as described above and instructions for use are provided. The article of manufacture comprises a container and a label or package insert on or associated with the container. Suitable containers include, for example, bottles, vials, syringes, IV solution bags, etc. The containers may be formed from a variety of materials such as glass or plastic. The container holds a pharmaceutical composition which is by itself or combined with another pharmaceutical composition is effective for treating, preventing and/or diagnosing the condition and may have a sterile access port. The composition may be contained in a transdermal, intradermal device for controlled sustained delivery of an antigen and AhR agonists.

The label or package insert indicates that the pharmaceutical composition is used for treating the condition of choice. The article of manufacture may comprise a container with a pharmaceutical composition contained therein, wherein the pharmaceutical composition comprises one or more antigens and at least one AhR agonists.

1. A method for inducing tolerance to an allergen in a subject, the method comprising administering to the subject, an effective amount of one or more antigens and at least one AhR agonists in a pharmaceutically acceptable form.

2. The method of claim 1, wherein the subject suffers from an allergic reaction upon contact with the allergen.

3. The method of claim 1, wherein the subject is suffering from an auto-immune disease.

4. The method of claim 1, wherein the antigen is selected from the group consisting of a polypeptide or protein, hapten, carbohydrate, nucleic acid, peptide, polyethylene glycol, lipid, polysaccharide, and gangliosides.

5. The method of claim 1, wherein the allergen is selected from the group consisting of an allergen derived from food, pollen, mold, pet dander, and dust mites.

6. The method of claim 1, wherein the antigen is an autoantigen.

7. The method of claim 1, wherein the administration of the effective amount of the one or more antigens and the AhR agonists is through the use of a slow-release device that releases said antigens and agonists in a sustained controlled manner.

8. The method of claim 7, wherein the slow-release device is a dermal patch.

9. The method of claim 7, wherein the slow-release device is an intradermal device that provides for the controlled sustained release of the one or more antigens and at least one AhR agonist into the body of a subject to be treated.

10. A pharmaceutical composition comprising an effective amount of (i) one or more antigens, wherein the antigen is an allergen selected from the group consisting of an allergen derived from food, pollen, mold, pet dander, and dust mites and (ii) at least one AhR agonists, wherein said AhR agonist is selected from the group consisting of Kynurenine and Kynurenic acid, in a pharmaceutically acceptable carrier.

11. (canceled)

12. (canceled)

13. The pharmaceutical composition of claim 10, wherein the one or more antigens and at least one AhR agonists are associated with a nanoparticle.

14. The pharmaceutical composition of claim 10, wherein the one or more antigens and at least one AhR agonists are associated with a liposome.

15. The pharmaceutical composition of claim 10, wherein the one or more antigens and at least one AhR agonists are associated with a slow-release device.

16. The pharmaceutical composition of claim 10, wherein the one or more antigens and at least one AhR agonists are associated with a dermal patch.

17. The pharmaceutical composition of claim 10, wherein the one or more antigens and at least one AhR agonists are associated with an intradermal delivery device that provides for the controlled sustained release of the one or more antigens and at least one AhR agonist into the body of a subject to be treated.

18. A kit comprising the pharmaceutical composition of claim 10 and a means for delivery into the body of a subject to be treated.

19. The kit of claim 18, wherein the pharmaceutical composition is associated with an intradermal delivery device.
